(19) United States
(12) Patent Application Publication
(10) Pub. No.: US 2025/0113854 A1
(43) Pub. Date: Apr. 10, 2025

(54) A DEVICE WITH BREATHING GUIDANCE MEANS

(71) Applicant: NICOVENTURES TRADING LIMITED, London (GB)

(72) Inventors: William ENGLAND, London (GB); Colin DICKENS, London (GB)

(21) Appl. No.: 18/836,406

(22) PCT Filed: Feb. 9, 2023
(86) PCT No.: PCT/GB2023/050291

§ 371 Date: Aug. 7, 2024

(30) Foreign Application Priority Data
Feb. 10, 2022 (GB) 2201748.7

(51) Int. Cl. A24B 15/167; A24B 15/24; A24F 40/42; A24F 40/485; A24F 40/65

(52) U.S. Cl. CPC A24B 15/167; A24B 15/243; A24F 40/42; A24F 40/485; A24F 40/65

(57) ABSTRACT

A device including a mouthpiece and an opening in fluid communication with the mouthpiece, wherein the device is configured to allow a user to breathe through the mouthpiece by drawing air in through the opening and expelling air out through the opening. The device further includes breathing guidance means configured to encourage compliance with a predetermined breathing regimen; and an air modifier. The air modifier includes any of: an air ionizer configured to ionize air drawn in through the opening; an aerosol generator for generating an aerosol for entrainment in air drawn in through the opening; a heater to heat air drawn in through the opening; or means to cool air drawn in through the opening.

### RELATED APPLICATIONS

The present application is a National Phase entry of PCT Application No. PCT/GB2023/050291 filed Feb. 9, 2023, which claims priority to GB Application No. 2201748.7 filed Feb. 10, 2022, each of which is hereby incorporated by reference in their entirety.

### FIELD OF THE INVENTION

The present invention relates to device with breathing guidance means.

### BACKGROUND

Taking control of our breathing can be useful to enhance a particular mood. For example, slowing the rate at which we breathe can have a calming effect, reducing anxiety; while other breathing patterns might be useful for improving concentration or increasing energy levels.

### SUMMARY OF THE INVENTION

In accordance with some embodiments described herein, in a first aspect there is provided a device comprising a mouthpiece and an opening in fluid communication with the mouthpiece, wherein the device is configured to allow a user to breathe through the mouthpiece by drawing air in through the opening and expelling air out through the opening, the device further comprising:
breathing guidance means configured to encourage compliance with a predetermined breathing regimen; and
an air modifier, wherein the air modifier comprises any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

In accordance with some embodiments described herein, in a second aspect there is provided a system comprising a handheld device and a mobile electronic device;
wherein the handheld device comprises a mouthpiece and an opening in fluid communication with the mouthpiece, the device being configured to allow a user to breathe through the mouthpiece by drawing air in through the opening and expelling air out through the opening, and wherein the device further comprises a flow rate sensor configured to generate data representing a flow rate of air expelled out through the opening, and breathing guidance means configured to encourage compliance with a predetermined breathing regimen;
wherein the mobile device is communicable with the handheld device and comprises computer code, the mobile device being configured to execute the computer code to receive data from the handheld device representing a flow rate of air expelled out through the opening; and wherein the handheld device further comprises an air modifier, the air modifier comprising any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

In accordance with some embodiments described herein, in a third aspect there is provided a method of operating a system comprising a handheld device and a mobile device;
wherein the handheld device comprises a mouthpiece and an opening in fluid communication with the mouthpiece, the device being configured to allow a user to breathe through the mouthpiece by drawing air in through the opening and expelling air out through the opening, and wherein the device further comprises a flow rate sensor configured to generate data representing a flow rate of air expelled out through the opening, and breathing guidance means configured to encourage compliance with a predetermined breathing regimen, the method comprising,
at the handheld device:
utilizing the flow rate sensor to generate data representing a flow rate of air expelled out through the opening,
transmitting the data representing the flow rate to the mobile device; and
at the mobile device:
receiving the data representing the flow rate of air expelled out through the opening;
   wherein the handheld device further comprises an air modifier, the air modifier comprising any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

In accordance with some embodiments described herein, in a fourth aspect there is provided a non-combustible aerosol provision device comprising an inlet and a mouthpiece in fluid communication with the inlet, wherein the device further comprises an adjustable restrictor configured to vary a draw resistance across the inlet.

### BRIEF DESCRIPTION OF THE DRAWINGS

Embodiments of the invention will now be described, by way of example only, with reference to accompanying drawings, in which:

FIG. 1 shows a schematic of a device according to embodiments of the invention;

FIG. 2 shows a schematic of a device according to embodiments of the invention;

FIG. 3 shows a schematic of an air modifier according to embodiments of the invention;

FIG. 4A shows a schematic of a device according to embodiments of the invention;

FIG. 4B shows a schematic of a device according to embodiments of the invention;

FIG. 5A shows a schematic of a device according to embodiments of the invention;

FIG. 5B shows a schematic of a device according to embodiments of the invention;

FIG. 6 shows a schematic of a device according to embodiments of the invention;

FIG. 7 shows a schematic of a device according to embodiments of the invention;

FIG. 8 is a schematic detail view showing an air modifier according to embodiments of the invention;

FIG. 9 is a schematic detail view showing an air modifier according to embodiments of the invention;

FIG. 10 is a schematic detail view showing an air modifier according to embodiments of the invention;

FIG. 11A is a top view of an article according to embodiments of the invention;

FIG. 11B is an end view of an article according to embodiments of the invention;

FIG. 11C is a side view of an article according to embodiments of the invention;

FIG. 12 is a section taken through an air modifier in accordance with embodiments of the invention;

FIG. 13 is a system comprising a device and an electronic mobile device according to embodiments of the invention;

FIG. 14 is a schematic of a device according to embodiments of the invention;

FIG. 15A is a top view of an article according to embodiments of the invention;

FIG. 15B is an end view of an article according to embodiments of the invention;

FIG. 15C is a side view of an article according to embodiments of the invention; and

FIG. 16 is a section taken through a device in accordance with embodiments of the invention.

### DETAILED DESCRIPTION

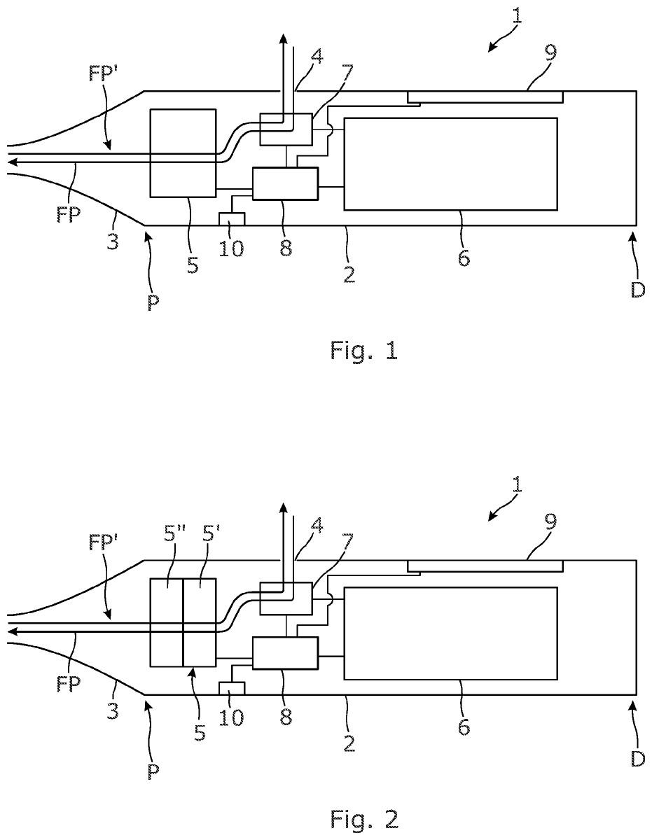

FIG. 1 is a schematic illustration of a handheld device 1 in accordance with embodiments described herein. The device 1 comprises an elongate housing 2 and a mouthpiece 3 provided at a proximal end P of the housing 2. The mouthpiece 3 fluidly communicates with an opening 4 in the housing 2 to allow a user of the device to breathe through the mouthpiece 3 by drawing air in through the opening 4 and exhaling air out through the opening 4.

A flow path FP, FP′ is defined between the opening 4 and the mouthpiece 3 along which air breathed in by, or exhaled by, the user is directed to travel. The flow path FP, FP′ comprising an inhalation flow path FP and an exhalation flow path FP′. An air modifier 5 is disposed in the inhalation flow path FP and is configured to modify the air drawn in through the opening 4. The air modifier 5 is configured to provide an additional quality to the air before it passes through the mouthpiece 3 for inhalation by the user.

The device 1 further comprises a power source 6, such as a battery 6. The battery 6 may be a rechargeable battery, such as a lithium ion battery. The battery 6 is configured to power the air modifier 5 and other associated systems as will be described below.

A flow sensor 7 is disposed across the flow path and is connected to both the battery 6, for power, and to a control unit 8. The flow sensor 7 is configured to detect when air is drawn in through the opening 4 and to send a signal to the control unit 8. In response to the signal, the control unit 8 distributes power to the air modifier 5 to modify the air drawn in through the opening 4. In some embodiments, the flow sensor 7 may comprise a pressure sensor 7. The pressure sensor 7 senses a drop in pressure across the opening 4 as a result of a user breathing in through the mouthpiece 3. It will be appreciated that other flow sensors 7 may be used, such a microphone or volumetric flow sensor.

In some embodiments, the air modifier 5 comprises an air ionizer 5 to ionize the air as it passes across the ionizer 5. Any air ionization technology may be employed, but by way of example, the air ionizer 5 may comprise an electrostatically charged surface. Electrostatic repulsion cause electrons to detach from the surface, attaching themselves to molecules of nitrogen and oxygen in the air, forming negative ions. In use, the air ionizer 5 is activated as a user breathes in through the mouthpiece 3 by applying voltage to the charged surface. Therefore, as the user inhales through the device 1, they breath in ionized air. A purported benefit of breathing ionized air is that it has a calming or anti-depressive effect.

In some embodiments, the air modifier 5 comprises means to cool air 5 drawn in through the inlet, so that the air is cooled as is passes through the air modifier and out through the mouthpiece. The means to cool air 5 may be a heat pump 5, as explained further below or, alternatively, the means to cool air 5 may be a passive cooling system 5. For example, the passive cooling system 5 may comprise an ice pack. In such an example, the air modifier is provided with a chamber to allow a user to insert an ice pack. The chamber may be accessible through an aperture in the housing 2 and comprise a cover replaceably insertable in the aperture to seal the chamber. The ice pack may contain water or any other nontoxic refrigerant. In use, the ice pack is first placed in a domestic freezer to cool the ice pack below the freezing temperature of the refrigerant. The ice pack is then removed from the freezer and inserted into the chamber of the device. When a user inhales through the device, the inhaled air is cooled as is passes through the air modifier 5 and over the ice pack. The ice pack may continue to provide a cooling effect until the temperature of the ice pack exceeds the melting point of the refrigerant, whereupon the ice pack may be removed and placed back in the freezer to allow the process to be repeated. The ice pack comprises a non-permeable shell to retain the refrigerant. Because the shell is non-permeable, the refrigerant does not leak out of the ice pack as it melts, preventing the refrigerant from entering air inhaled by the user.

In some embodiments, the air modifier 5 comprises a heater 5′ and a heat pump 5″, as schematically illustrated by FIG. 2. It will be appreciated that using both a heater 5′ and a heat pump 5″ allows the user to selectively heat or cool the air drawn through the device according to whether the heater 5′ or heat pump 5″ is activated. In one embodiment the heater 5′ comprises a filament heater 5′. As a user breathes in through the mouthpiece, power from the power source induces a current in the filament heater 5′ to increase its temperature. The filament therefore heats the air as it passes over the filament and travels along the flow path.

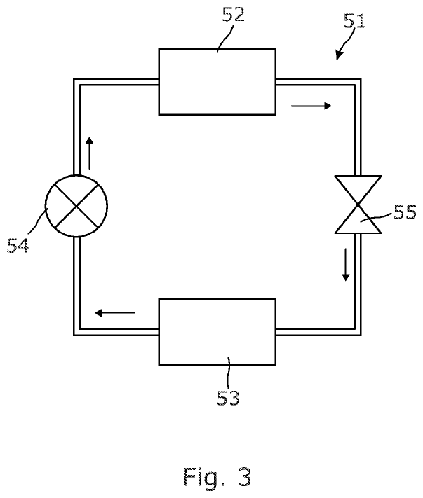

The heat pump 5″ is configured to cool air passing along the flow path. As a user breathes in through the mouthpiece 3, power from the power source is sent to the heat pump 5″, activating the heat pump 5″. The heat pump 5″ operates on a conventional refrigeration cycle (illustrated in FIG. 3 for reference) and comprises a refrigerant circuit 51, a condenser 52, an evaporator 53, a pump/compressor 54 and an expansion valve 55. Refrigerant is expanded through the expansion valve 55 into the evaporator 53 taking up heat, whereupon it travels to a compressor 54 for compression and then to the condenser 52 for condensing and heat rejection. The evaporator 53 is disposed in the flow path FP to cool air as it passes across the evaporator 53.

It shall be appreciated that in some embodiments, the air modifier 5 comprises one of a heater or a heat pump. It is not a requirement that both a heater and heat pump are provided.

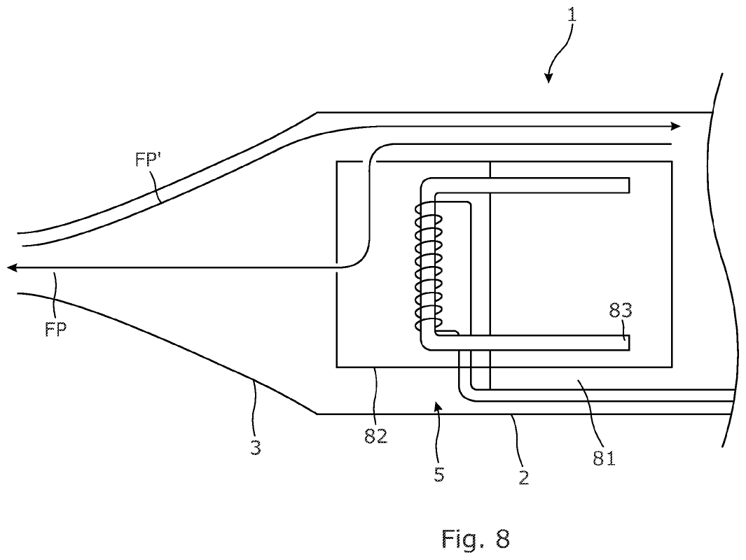

In some embodiments, the air modifier comprises a non-combustible aerosol provision system 5. Example non-combustible aerosol provision systems 5 are illustrated by FIGS. 8 to 10. According to the present disclosure, a “non-combustible” aerosol provision system is one that facilitates delivery of at least one substance to a user, without a constituent aerosol-generating material being burned or combusted. It will be appreciated that, to allow for an enlarged illustration, each of the aerosol provision systems 5 of FIGS. 8 to 10 are shown absent of any features of the device 1 distal to the aerosol provision system 5. However, the aerosol provision systems 5 of FIGS. 8 to 10 may be installed in any of the devices 1 of the embodiments described and illustrated herein. It will be appreciated that the aerosol provision systems 5 of FIGS. 8 to 10 may be installed where the air modifier 5 is schematically shown in FIG. 1, 2, 6 or 7. By ‘distal to’, it is meant closer to the distal end D of the housing 2. The distal end D is the end of the housing 2 opposite the proximal end P mentioned above.

In some embodiments, the non-combustible aerosol provision system 5 comprises an electronic cigarette, also known as a vaping device or electronic nicotine delivery system (END), although it is noted that the presence of nicotine in the aerosol-generating material is not a requirement. The aerosol generating material is typically an aerosol former material. In such embodiments, an example of which is illustrated by FIG. 8, the device 1 may comprise a tank 81 for storing liquid aerosol former material, a heating chamber 82 for heating and vaporizing the former material and a wick 83 for drawing the former material into the heating chamber 82. A heating element 84, typically a resistive coil, is disposed in proximity to the wick 83. In use, power is delivered to the heating element 84 in response to a user breathing in through the device 1 to vaporize aerosol former material drawn into the heating chamber 82 by the wick 83. Specifically, when the flow sensor 7 detects a pressure drop across the opening 4, the control unit 8 distributes power from the battery 6 to the heating element 84. The inhalation flow path FP is directed through the heating chamber 82 so that vaporized aerosol former material is entrained into the flow path FP for inhalation by the user. Aerosol former material can be replaced as it is consumed by refilling the tank 81, once depleted. In some embodiments, the tank 81 itself is removable from the device so that it can be disposed of and replaced when depleted. In some embodiments, the whole assembly 5 of tank 81, chamber 82, wick 83 and heating element 84 can be removed and replaced. Such removable assemblies are often known as cartomizers.

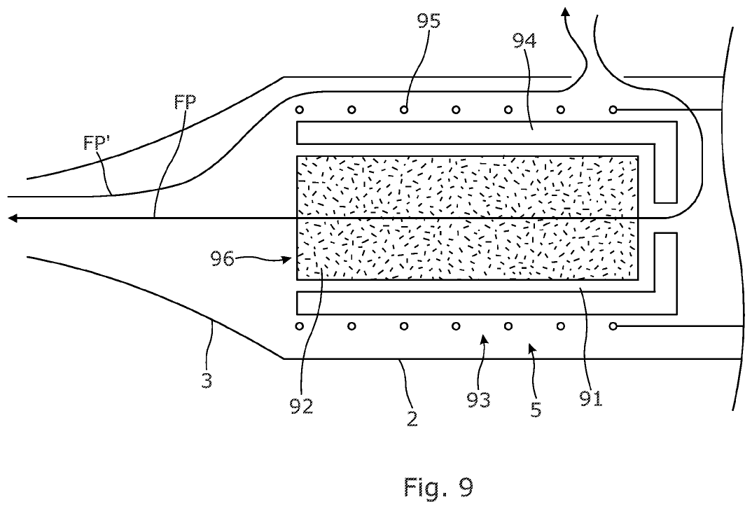

In some embodiments, the non-combustible aerosol provision system 5 comprises an aerosol-generating material heating system 5, also known as a heat-not-burn system. An example of such a tobacco heating system 5 is illustrated by FIG. 9. The system comprises a heating chamber 90 for receiving tobacco material 92 and a heater 93 for heating the heating chamber 90. In this example, the heater 93 comprises an induction heating system having a susceptor 94 and an induction coil 95. The susceptor 94 is a wall of the heating chamber 90, the induction coil 95 being arranged radially around the heating chamber 90 to heat the susceptor 94 by a varying magnetic field. In use, power is delivered to the induction coil 95 in response to a user breathing in through the device. Specifically, when the flow sensor 7 detects a pressure drop across the opening 4, the control unit 8 distributes power from the battery to the induction coil 95 which then generates a varying magnetic field to heat the susceptor 94. The tobacco material 92 received in the heating chamber 90 is heated by the susceptor 94 so that volatile elements of the tobacco 92 are released and entrained into the flow path FP for inhalation by the user. Typically, the tobacco material 92 is provided in a consumable 96 which is inserted into the heating chamber 90 prior to use of the device 1. Once the consumable 96 has expired, it can be removed for replacement. In the illustrated example, the mouthpiece 3 can be removed for access to the heating chamber 90 and the insertion and removal of consumables 96.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

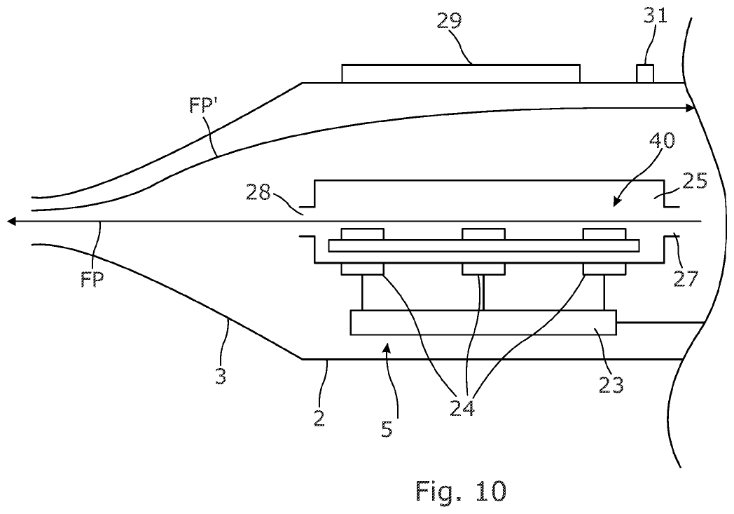

In some embodiments, the non-combustible aerosol provision system is configured to heat aerosol generating material provided on a carrier component, as in the example shown in FIG. 10.

The aerosol provision system of FIG. 10 comprises control circuitry 23, a plurality of aerosol generating components 24, a receptacle or aerosol forming chamber 25, an air inlet 27, an air outlet 28, an activation button 29 and an end of use indicator 31.

The control circuitry 23 is configured to control the aerosolization of aerosol generating material, as described in more detail below. The control circuitry 23 is connected to the battery 6 and the control unit 8.

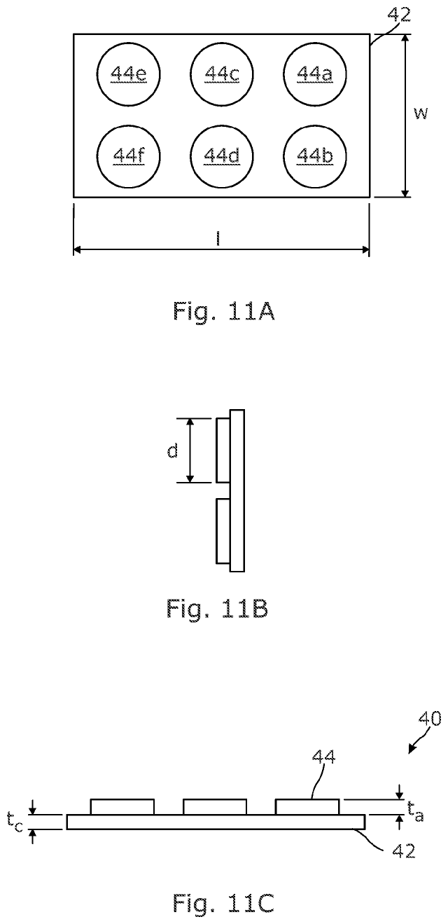

The receptacle 25 is arranged to receive an aerosol generating article 4. The aerosol generating article 40 comprises a carrier component 42 and aerosol generating material 44. An example aerosol generating article 4 having a carrier component is shown in more detail in FIGS. 11A to 11C. FIG. 11A is a top-down view of the article 40, FIG. 11B is an end-on view along the longitudinal (length) axis of the article 40, and FIG. 11C is a side-on view along the width axis of the article 40.

In this example, the carrier component 42 is formed of card. The carrier component 42 forms the majority of the article 40, and acts as a base for the aerosol generating material 44 to be deposited on. The carrier component 42 is broadly cuboidal in shape has a length l, a width w and a 20 thickness tc as shown in FIGS. 11A to 11C. By way of example, the length of the carrier component 42 may be 30 to 80 mm, the width may be 7 to 25 mm, and the thickness may be between 0.2 to 1 mm. However, it should be appreciated that the above are exemplary dimensions of the carrier component 42, and in other implementations the carrier component 42 may have different dimensions as appropriate. In the illustrated example, the article 40 comprises a plurality of discrete portions of aerosol generating material 44 disposed on a surface of the carrier component 42. More specifically, the article 40 comprises six discrete portions of aerosol generating material 44, labelled 44a to 44f, disposed in a two by three array. However, it should be appreciated that in other implementations a greater or lesser number of discrete portions may be provided, and/or the portions may be disposed in a different array (e.g., a one by six array). In the example shown, the aerosol generating material 44 is disposed at discrete, separate locations on a single surface of the component carrier 42. The discrete portions of aerosol generating material 44 are shown as having a circular footprint, although it should be appreciated that the discrete portions of aerosol generating material 44 may take any other footprint, such as square or rectangular, as appropriate. The discrete portions of aerosol generating material 44 have a diameter d and a thickness ta as shown in FIGS. 11A to 11C. The thickness ta may take any suitable value, for example the thickness ta may be in the range of 50 μm to 1.5 mm. In some embodiments, the thickness ta is from about 50 μm to about 200 μm, or about 50 μm to about 100 μm, or about 60 μm to about 90 μm, suitably about 77 μm. In other embodiments, the thickness ta may be greater than 200 μm, e.g., from about 50 μm to about 400 μm, or to about 1 mm, or to about 1.5 mm.

The discrete portions of aerosol generating material 44 are separate from one another such that each of the discrete portions may be energised (e.g., heated) individually or selectively to produce an aerosol. In some implementations, the portions of aerosol generating material 44 may have a mass no greater than 20 mg, such that the amount of material to be aerosolized by a given aerosol generating component 24 at any one time is relatively low. For example, the mass per portion may be equal to or lower than 20 mg, or equal to or lower than 10 mg, or equal to or lower than 5 mg. Of course, it should be appreciated that the total mass of the article 40 may be greater than 20 mg.

In the described implementation, the aerosol generating material 44 is an amorphous solid. Generally, the amorphous solid may comprise a gelling agent (sometimes referred to as a binder) and an aerosol generating agent (which might comprise glycerol, for example). Optionally, the aerosol generating material may comprise one or more of the following: an active substance (which may include a tobacco extract), a flavorant, an acid, and a filler. Other components may also be present as desired.

An amorphous solid aerosol generating material offers some advantages over other types of aerosolizable materials commonly found in some electronic aerosol provision devices. For example, compared to electronic aerosol provision devices which aerosolize a liquid aerosolizable material, the potential for the amorphous solid to leak or otherwise flow from a location at which the amorphous solid is stored is greatly reduced. This means aerosol provision devices or articles may be more cheaply manufactured as the components do not necessarily require the same liquid-tight seals or the like to be used.

The article 40 may comprise a plurality of portions of aerosol generating material all formed form the same aerosol generating material (e.g., one of the amorphous solids described above). Alternatively, the article 40 may comprise a plurality of portions of aerosol generating material 44 where at least two portions are formed from different aerosol generating material (e.g., one of the amorphous solids described above).

The receptacle 25 is suitably sized to removably receive the article 40 therein. Although not shown in FIG. 10, the device 1 may comprise a hinged door or removable part of the housing 2 to permit access to the receptacle 25 such that a user may insert and/or remove the article 40 from the receptacle 25. The hinged door or removable part of the housing 2 may also act to retain the article 40 within the receptacle 25 when closed. When the aerosol generating article 40 is exhausted or the user simply wishes to switch to a different aerosol generating article 40, the aerosol generating article 40 may be removed from the device 1 and a replacement aerosol generating article 40 positioned in the receptacle 25 in its place.

As seen in FIG. 10, the aerosol provision system 5 comprises a number of aerosol generating components 24. In this example, the aerosol generating components 24 are heating elements 24, and more specifically resistive heating elements 24. Resistive heating elements 5 24 receive an electrical current and convert the electrical energy into heat. The resistive heating elements 24 may be formed from, or comprise, any suitable resistive heating material, such as NiChrome (Ni20Cr80), which generates heat upon receiving an electrical current.

FIG. 12 is a cross-sectional, top-down view of the aerosol provision system 5 of FIG. 10, showing the arrangement of the heating elements 24 in more detail. In FIGS. 10 and 12, the heating elements 24 are positioned such that a surface of the heating element 24 forms a part of the surface of the receptacle 25. That is, an outer surface of the heating elements 24 is flush with the inner surface of the receptacle 25. More specifically, the outer surface of the heating element 24 that is 15 flush with the inner surface of the receptacle 25 is a surface of the heating element 24 that is heated (i.e., its temperature increases) when an electrical current is passed through the heating element 24.

The heating elements 24 are arranged such that, when the article 40 is received in the receptacle 25, each heating element 24 aligns with a corresponding discrete portion of aerosol generating material 44. Hence, in this example, six heating elements 24 are arranged in a two by three array broadly corresponding to the arrangement of the two by three array of the six discrete portions of aerosol generating material 44 shown in FIGS. 11A to 11C. However, as discussed above, the number of heating elements 24 may be different in different implementations, for example there may be 8, 10, 12, 14, etc. heating elements 24. In some implementations, the number of heating elements 24 is greater than or equal to six but no greater than 20. More specifically, the heating elements 24 are labelled 24a to 24f in FIG. 12, and it should be appreciated that each heating element 24 is arranged to align with a corresponding portion of aerosol generating material 44 as denoted by the corresponding letter following the references 24/44. Accordingly, each of the heating elements 24 can be individually activated to heat a corresponding portion of aerosol generating material 44. While the heating elements 24 are shown flush with the inner surface of the receptacle 25, in other implementations the heating elements 24 may protrude into the receptacle 25. In either case, the article 40 contacts the surfaces of the heating elements 24 when present in the receptacle 25 such that heat generated by the heating elements 24 is conducted to the aerosol generating material 44 through the carrier component 42.

In use, the device 1 (and more specifically the control circuitry 23) is configured to deliver power to the heating elements 24 in response to a user breathing in through the device 1. When the flow sensor 7 detects a pressure drop across the opening 4, the control unit 8 distributes power from the battery to the control circuitry 23. The control circuitry 23 is configured to selectively apply power to the heating elements 24 to subsequently heat the corresponding portions of aerosol generating material 44 to generate aerosol. In particular, the control circuitry 23 may be configured to receive signalling from the control unit 8 and to use this signalling to determine if a user is inhaling on the device 1. If the control circuitry 23 receives this signalling, then the control circuitry 23 is configured to supply power from the power source 6 to one or more of the heating 20 elements 24. Power may be supplied for a predetermined time period (for example, three seconds) from the moment inhalation is detected, or in response to the length of time the inhalation is detected for. Therefore, when a user breathes in through the device 1, air is drawn into the opening 4 and in through the air inlet 27 of the receptacle 25 where it mixes with the aerosol generated by heating the aerosol generating material 44, and then to the user's mouth via air outlet 28.

In some embodiments, the activation button 29 can be pressed by a user to start aerosol generation. The control circuitry 23 may be configured to receive signalling from the button 29 to determine if a user is pressing the button 29. If the control circuitry 23 receives this signalling, then the control circuitry 23 is configured to supply power from the power source 6 to one or more of the heating elements 24. Power may be supplied for a predetermined time period (for example, three seconds) from the signalling is detected, or in response to the length of time the signalling is detected for. In other implementations, the button 29 may be replaced by a screen type user interface.

In some implementations, in response to detecting the signalling from either one or both of the button 29 and control unit 8, the control circuitry 23 is configured to sequentially supply power to each of the individual heating elements 24. For example, the control circuitry 23 may be configured to supply power to a first heating element 24 of the plurality of heating elements 24 when the signalling is first detected (e.g., from when the device 1 is first switched on). When the signalling stops, or in response to a predetermined time from the signalling being detected elapsing, the control circuitry 23 registers that the first heating element 24 has been activated (and thus the corresponding discrete portion of aerosol generating material 44 has been heated). The control circuitry 23 determines that in response to receiving subsequent signalling from either one or both of the button 29 and control unit 8 is received by the control circuitry 23, the control circuitry 23 activates the second heating element 24. This process is repeated for remaining heating elements 24, such that all heating elements 24 are sequentially activated. Effectively, this operation means that for each inhalation a different one of the discrete portions of aerosol generating material 44 is heated and an aerosol generated therefrom. In other words, a single discrete portion of aerosol generating material is heated per user inhalation.

The device 1 of embodiments described herein further comprises breathing guidance means 9. The breathing guidance means 9 is configured to encourage compliance with a predetermined breathing regimen. By ‘predetermined breathing regimen’ it is meant a breathing cycle having a predetermined inhalation and exhalation duration and/or flow rate. The control unit 8 utilizes information generated by the flow sensor 7 to determine variation from the predetermined breathing regimen and, in turn, operates the breathing guidance means 9 to encourage a breathing pattern closer to the predetermined breathing regimen.

In some embodiments, the flow sensor 7 is configured to determine the direction of flow through the opening 4 and to send a signal to the control unit 8. The signal will indicate the direction of flow through the opening 4 and, therefore, whether a user is inhaling or exhaling through the device 1. The flow sensor 7 will generate the signal for the duration of the inhalation or exhalation. The control unit 8 compares, in real time, the duration of the signal to a predetermined duration of inhalation or exhalation consistent with the predetermined breathing regimen. The control unit 8 continuously calculates the time elapsed since the signal was first received as a percentage of the predetermined duration. The control unit 8 sends a signal representative of the time elapsed since the signal was first received to the breathing guidance means 9 at predetermined intervals. The predetermined intervals correspond to predetermined percentages of the predetermined duration. For example, the control unit 8 may be configured to send a signal to the breathing guidance means representative of the duration of inhalation or exhalation having reached 20%, 40%, 60%, 80% and 100% of the predetermined duration. The breathing guidance means 9 is configured to convey to the user of the device that their inhalation or exhalation has reached said predetermined percentages of the predetermined duration and thereby encourage compliance with the predetermined breathing regimen.

In some embodiments, the breathing guidance means 9 comprises visual means 9. The visual means may comprise a series of LED lights 91 or a screen 92 provided in the housing 2 of the device 1. The visual means 9 are configured to indicate to a user of the device whether they are in compliance with a predetermined breathing regimen.

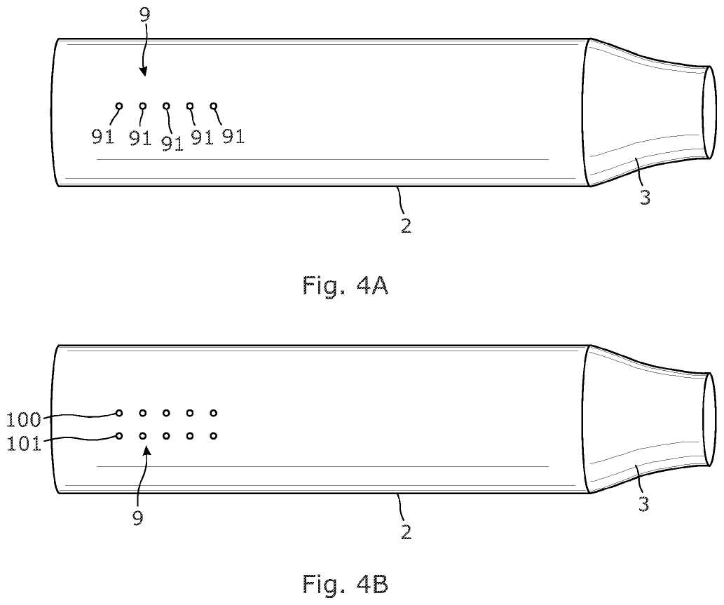

In one embodiment illustrated by FIG. 4A, the visual means 9 comprises a series of LEDs 91 that illuminate in sequence as a user breathes through the device. Five LEDs are illustrated, though it will be appreciated that more or less LEDs may be used as appropriate. The breathing guidance means 9 is configured to illuminate an LED on each occasion that a signal is received from the control unit 8 representative of the duration of inhalation or exhalation. For example, as a user breathes in through the device 1, the control unit 8 sends a signal to the breathing guidance means to illuminate an LED on each occasion that the duration of inhalation is equal to 20%, 40%, 60%, 80% and 100% of the predetermined duration. The LEDs are illuminated in sequence, with illumination of one LED corresponding to the duration of inhalation equaling 20% of the predetermined duration, illumination of two LEDs corresponding to the duration of inhalation equaling 40% of the predetermined duration and so on, until all five of the LEDs are illuminated to signify that duration of inhalation was at least equal to 100% of the predetermined duration. The user is therefore encouraged to inhale for the predetermined duration by visual inspection of the number of LEDs illuminated. The user can also monitor their progress toward 100% of the predetermined duration in real time by watching the LEDs illuminate in turn. Exactly the same process is employed during exhalation. As the user breathes out through the device, the control unit 8 sends a signal to the breathing guidance means to illuminate an LED on each occasion that the duration of exhalation is equal to 20%, 40%, 60%, 80% and 100% of the predetermined duration. The LEDs are again illuminated in sequence, with illumination of one LED corresponding to the duration of exhalation equaling 20% of the predetermined duration, illumination of two LEDs corresponding to the duration of exhalation equaling 40% of the predetermined duration and so on, until all five of the LEDs are illuminated to signify that duration of exhalation was at least equal to 100% of the predetermined duration. As before, the user is encouraged to exhale for the predetermined duration by visual inspection of the number of LEDs illuminated and can monitor their progress toward 100% of the predetermined duration in real time by watching the LEDs illuminate in turn. Each time the user transitions from inhalation to exhalation, or vice versa, the LEDs are reset to all off, so that the prevailing inhalation or exhalation event can be monitored. The user is therefore able to determine the duration of their inhalation or exhalation relative to the predetermined breathing regimen.

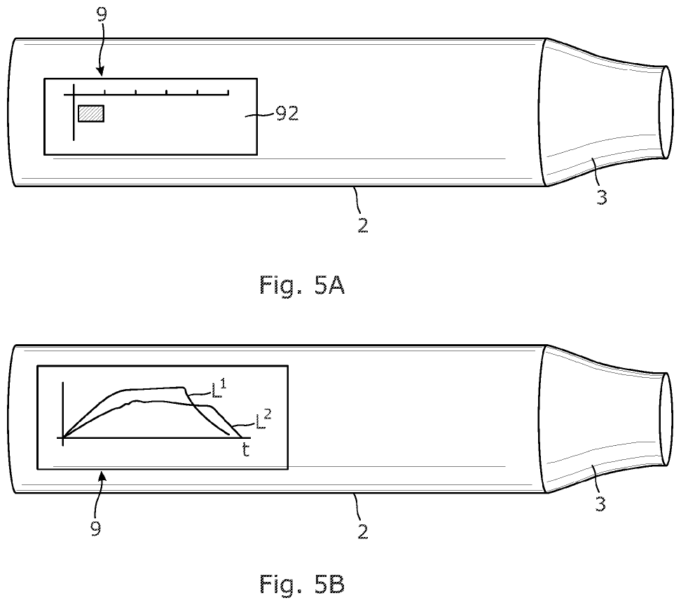

Instead of LEDs, the visual means can comprise an LCD screen or similar. This is illustrated by FIG. 5. The breathing guidance means 9 is configured to adjust the display on each occasion that a signal is received from the control unit 8 representative of the duration of inhalation or exhalation. For example, as a user breathes in through the device 1, the control unit 8 sends a signal to the breathing guidance means to adjust the display on each occasion that the duration of inhalation is equal to 20%, 40%, 60%, 80% and 100% of the predetermined duration. The display may comprise a bar graph, with the percentage of the predetermined duration on the Y axis. The bar progresses over the duration of inhalation. For example, the display is adjusted so that the bar graph reads 20% when the breathing guidance means 9 receives a signal corresponding to the duration of inhalation equaling 20% of the predetermined duration, the display being further adjusted so that the bar graph reads 40% when the breathing guidance means 9 receives a signal corresponding to the duration of inhalation equaling 40% of the predetermined duration and so on, until the bar graph displays 100% of the predetermined duration. The user is therefore encouraged to inhale for the predetermined duration by visual inspection of the display. The user can also monitor their progress toward 100% of the predetermined duration in real time by watching the progress of the display. Exactly the same process is employed during exhalation. As a user breathes out through the device 1, the control unit 8 sends a signal to the breathing guidance means to adjust the display on each occasion that the duration of exhalation is equal to 20%, 40%, 60%, 80% and 100% of the predetermined duration. The bar progresses over the duration of exhalation. As in the above example, the display may be adjusted so that the bar graph reads 20% when the breathing guidance means 9 receives a signal corresponding to the duration of exhalation equaling 20% of the predetermined duration, the display being further adjusted so that the bar graph reads 40% when the breathing guidance means 9 receives a signal corresponding to the duration of exhalation equaling 40% of the predetermined duration and so on, until the bar graph displays 100% of the predetermined duration. The user is therefore encouraged to exhale for the predetermined duration by visual inspection of the display. The user can also monitor their progress toward 100% of the predetermined duration in real time by watching the progress of the display. Each time the user transitions from inhalation to exhalation, or vice versa, the display is adjusted to reset the bar graph, so that the prevailing inhalation or exhalation event can be monitored. The user is therefore able to determine the duration of their inhalation or exhalation relative to the predetermined breathing regimen.

It shall be appreciated that the advantage of using a display over LED lights is that greater resolution can be more easily obtained. By ‘greater resolution’ it is meant that, for either inhalation or exhalation, the level of progress toward 100% of the predetermined duration can be resolved to a smaller degree. To achieve higher resolution using LED lights, more LED lights would be required, which is not always practicable. Using a display allows any information to be displayed and, therefore, any level of resolution. For example, the control unit 8 may be configured to send a signal to the breathing guidance means 9 representative of the duration of inhalation or exhalation having reached any percentage of the predetermined duration. The display may be configured to display this information as smaller increments of progression on a bar graph, or by displaying the progression numerically, that is to say, displaying 20% progression as ‘20%’ and so on.

In embodiments described herein, the flow sensor 7 may be further configured to determine the flow rate through the opening 4 and to send a signal to the control unit 8. The signal will indicate the flow rate through the opening 4. The skilled person will appreciate that the measured flow rate can be either a volumetric or mass flow rate depending on the type of sensing equipment used. The flow sensor 7 will generate the signal for the duration of the inhalation or exhalation. The signal amplitude will correspond to the flow rate. The control unit 8 compares, in real time, the amplitude of the signal to a predetermined amplitude consistent with the predetermined breathing regimen. The control unit 8 continuously calculates the amplitude of the signal as a percentage of the predetermined amplitude. The control unit 8 may therefore send a signal representative of the percentage the predetermined amplitude to the breathing guidance means 9.

Where the breathing guidance means 9 comprises five LEDs 91 as shown in FIG. 4A, the device 1 may utilize the LEDs 91 to indicate to a user whether their breathing flow rate (i.e. how hard they are breathing) matches the predetermined breathing regimen. In such embodiments, the control unit 8 resolves the percentage of the predetermined amplitude to one of five levels. In other words, the control unit 8 resolves the percentage of the predetermined amplitude into one of five bands comprising: <50%, 51%-90%, 91%-110%, 111%-150% or >150%. The control unit 8 sends a signal to the breathing guidance means 9 that is representative of one of the five bands. The breathing guidance means 9 is configured to illuminate a pattern of LEDs 91 corresponding to the signal received from the control unit 8. In one embodiment, the LEDs 91 illuminate in sequence as the signal value indicates an increase from one band to the next. Therefore, starting from one end of the line of LEDs 91, one illuminated LED 91 indicates that the flow rate of the user breathing through the device is <50% of the predetermined flow rate; two consecutive LEDs 91 indicates that the flow rate of the user breathing through the device 1 is between 51% and 90% of the predetermined flow rate and so on. Therefore, when three LEDs 91 are illuminated, the user knows that flow rate is within an acceptable margin of the predetermined flow rate. More than three LEDs 91 indicates that the flow rate exceeds the predetermined flow rate. The number of illuminated LEDs 91 will indicate how ‘hard’ the user is inhaling or exhaling through the device. In one embodiment, the LEDs 91 can be colored to further signify whether the user is breathing too hard or too soft. For example, a traffic light system can be used, with the first and fifth LED 91 being red, the second and fourth LED 91 being amber and the third (central) LED 91 being green. The user is therefore encouraged to control their breathing by visual inspect of the display 9 to encourage compliance with the predetermined breathing regimen.

Clearly, if the LEDs 91 are being used to indicate to the user how ‘hard’ they are breathing (i.e. the flow rate of inhalation and exhalation) relative to a predetermined breathing regimen, they are not available for use to indicate the duration of their inhalation or exhalation relative to the predetermined breathing regimen. Therefore, the device 1 may be further provided with a switch so that the user can switch between a first mode in which the LEDs 91 are used to indicate how hard or soft they are breathing relative to the predetermined breathing regimen, and a second mode in which the LEDs 91 are used to indicate the duration of their inhalation or exhalation relative to the predetermined breathing regimen. Alternatively, the breathing guidance means may comprise two rows of LEDs as shown in FIG. 4B, each row being used in the manner described above. Therefore a first row 100 is used to indicate how hard the user is breathing relative to the predetermined breathing regimen and a second row 101 is used to indicate the duration of their inhalation or exhalation relative to the predetermined breathing regimen. This means the user can monitor both at the same time.

In embodiments in which the visual means comprises an LCD screen, the user may be simultaneously informed of how hard they are breathing and the duration of their inhalation or exhalation relative to the predetermined breathing regimen. Such information can be delivered simultaneously by any appropriate graphical indication. In one embodiment, shown in FIG. 5B, the LCD screen comprises a graph with flow rate on the Y axis and time on the X axis. The graph comprises a line 11 that represents a predetermined breathing regimen. As the user breathes through the device, the display is configured to trace a line 12 that represents the user's breathing flow rate with time. By attempting to ensure that the traced line 12 follows the line 11 representative of the predetermined breathing regimen, the user is encouraged to comply with the predetermined breathing regimen as they breathe through the device 1. Each time the user transitions from inhalation to exhalation, or vice versa, the display 9 is adjusted to reset and the line is traced afresh.

It will be appreciated that the predetermined breathing regimen is selected to promote a preferred mood. In one embodiment, the predetermined breathing regimen may be selected to promote a relaxed state. For example, the predetermined breathing regimen may comprise an inhalation to exhalation ratio of 1:2. That is, the predetermined breathing regimen may comprise an inhalation period that is half the exhalation period. For example, the inhalation period may be 2 to 4 second, while the exhalation period may be 4 to 8 seconds.

In some embodiments, the predetermined breathing regimen may also comprise a hold period. By ‘hold period’ it is meant a period of time in which the user is encouraged to hold their breath between inhalation and exhalation. For example, the predetermined breathing regimen may comprise a ratio of inhalation, hold and exhalation of 4:7:8. That is, the inhalation period is four sevenths of the hold period and four eighths (half) of the exhalation period. For example, the inhalation period may be 4 seconds, the hold period 7 seconds and the exhalation period 8 seconds. In embodiments such as the embodiment of FIGS. 4A and 4B—where the breathing guidance means comprises LEDs 91—the beginning of the hold period may be indicated by all of the LEDs illuminating simultaneously, and a countdown to the end of the hold period may be visually represented by an LED 91 being extinguished for each one fifth of the passing hold period (where five LEDs are employed). In embodiments such as the embodiment of FIG. 5—where a display is used-a message may be shown on the display indicating to the user that they are to hold their breath. The message may be accompanied by a timer counting down to the end of the hold period.

In some embodiments, the hold period is triggered by a signal from the control unit 8 that indicates that the predetermined inhalation period has elapsed and the hold period has begun.

In some embodiments, the breathing guidance means 9 comprises haptics or audio in addition, or in place of, the visual means.

In one embodiment, the breathing guidance means 9 comprises a haptic motor 9. The haptic motor 9 is configured so that a user of the device can feel haptic feedback generated by the haptic motor through contact with the housing 2 of the device 1. The haptic feedback may comprise a pulse of vibration.

In one embodiment, the haptic motor 9 is configured to provide haptic feedback when the control unit 8 sends a signal to the breathing guidance means 9 representative of the duration of inhalation or exhalation having reached 100% of the predetermined duration. In one embodiment, the haptic motor 9 is configured to provide haptic feedback at the start and end of the hold period.

In one embodiment, the breathing guidance means 9 comprises a speaker 9 to provide audio feedback. The speaker is configured so that a user of the device can hear audio feedback generated by the speaker 9 while breathing through the device 1. The audio feedback may comprise a sound or musical note.

In one embodiment, the speaker 9 is configured to provide audio feedback when the control unit 8 sends a signal to the breathing guidance means representative of the duration of inhalation or exhalation having reached 100% of the predetermined duration. In one embodiment, the speaker 9 is configured to provide audio feedback at the start and end of the hold period.

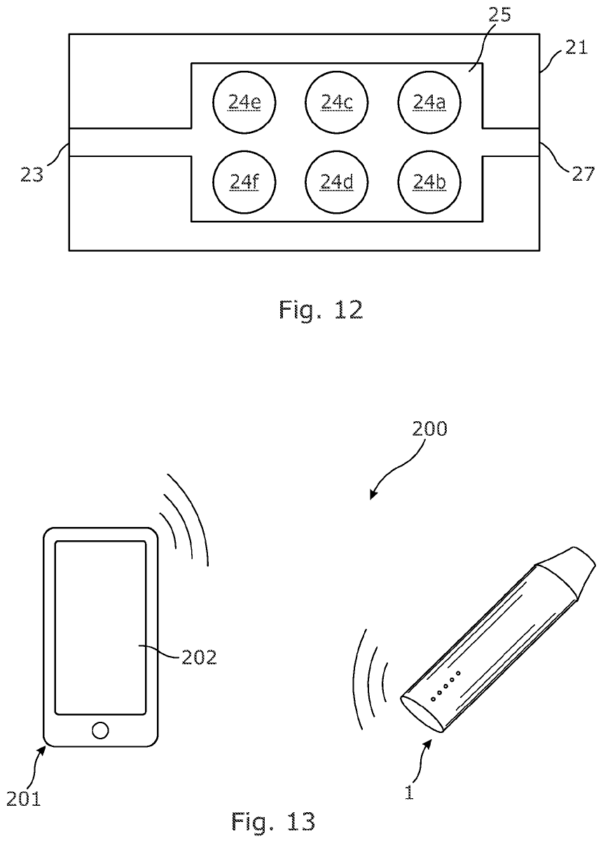

In some embodiments, the breathing guidance means 9 comprises a mobile electronic device 201. A system 200 comprising the device 1 and the mobile electronic device 201 is illustrated in FIG. 13. The mobile electronic device 201 may be in addition, or in place of, the haptics, audio or visual means. The mobile electronic device 201 may be a smartphone, tablet or similar. The mobile device electronic 201 is communicable with the handheld device and comprises computer code that, when executed by the mobile device, receives data from the handheld device 1. In one embodiment, the data represents the direction of flow through the opening and/or the flow rate of flow through the opening.

In order to facilitate communication with a mobile electronic device 201, the handheld device 1 is further provided with a transmitter 10 electrically connected to the control unit. The transmitter 10 is configured to transmit data wirelessly to the mobile electronic device 201. The transmitter 10 may comprise a Bluetooth transmitter 10 to communicate directly with the mobile electronic device 201, or the transmitter 10 may be configured to communicate with a wireless network of which the mobile electronic device 201 is a part.

As discussed above, the flow sensor 7 determines the direction of flow through the opening 4 and sends a signal to the control unit 8. The signal indicates the direction of flow through the opening 4 and, therefore, whether a user is inhaling or exhaling through the device 1. The flow sensor 7 generates the signal for the duration of the inhalation or exhalation which the control unit 8 converts to data available for transmission by the transmitter 10. The mobile device executes the computer code to obtain the data, which is transmitted continuously for the period of inhalation or exhalation. The computer code of the mobile device 201 is further executed to compare, in real time, the duration of the signal to a predetermined duration of inhalation or exhalation consistent with the predetermined breathing regimen. The computer code runs a continuous calculation of the time elapsed since the data of particular flow direction was first transmitted as a percentage of the predetermined duration. The results of this calculation are duration data. The duration data are then displayed to the user of the handheld device via a display 202 of the mobile electronic device 201; in the same way that the equivalent data are displayed on the device 1 itself, as shown in FIG. 5A. Therefore, the display comprises a bar graph, with the percentage of the predetermined duration on the Y axis. The bar progresses over the duration of inhalation or exhalation. The user is therefore encouraged to inhale or exhale for the predetermined duration by visual inspection of the display. The user can also monitor their progress toward 100% of the predetermined duration in real time by watching the progress of the display. It will be appreciated that, in the alternative to the duration data being calculated at the mobile electronic device 201, it may instead be calculated by the control unit 8 of the handheld device 1 and converted to data available for transmission to the mobile electronic device 201. In such examples, the mobile electronic device 201 executes the computer code only for the purpose of displaying the duration data in any of the ways described.

Also as above, the flow sensor 7 may determine the flow rate through the opening 4 and send a signal to the control unit 8. The signal will indicate the flow rate through the opening 4 as a volumetric or mass flow. The flow sensor 7 will generate the signal for the duration of the inhalation or exhalation which the control unit 8 converts to data available for transmission by the transmitter 10. The mobile device 201 executes the computer code to obtain the data, which is transmitted continuously for the period of inhalation or exhalation. The computer code of the mobile device 201 is further executed to compare, in real time, the measured flow rate to the predetermined flow rate and output this as a percentage. The results of this calculation are flow rate data. The flow rate data are then displayed to the user of the handheld device 1 via the display 202 of the mobile electronic device 201. For example, the display may combine the duration and flow rate data by displaying a graph with flow rate on the Y axis and time on the X axis; in the same way that the equivalent data are displayed on the device 1 itself, as shown in FIG. 5B. Therefore, the graph comprises a line that represents a predetermined breathing regimen and, as the user breathes through the device, the display 202 is configured to trace a line that represents the user's breathing flow rate with time. By attempting to ensure that the traced line follows the line representative of the predetermined breathing regimen, the user is encouraged to comply with the predetermined breathing regimen as they breathe through the device 1. Each time the user transitions from inhalation to exhalation, or vice versa, the display 202 is adjusted to reset and the line is traced afresh. It will be appreciated that, in the alternative to the flow rate data being calculated at the mobile electronic device 201, it may instead be calculated by the control unit 8 of the handheld device 1 and converted to data available for transmission to the mobile electronic device 201. In such examples, the mobile electronic device 201 executes the computer code only for the purpose of displaying the flow rate data in any of the ways described.

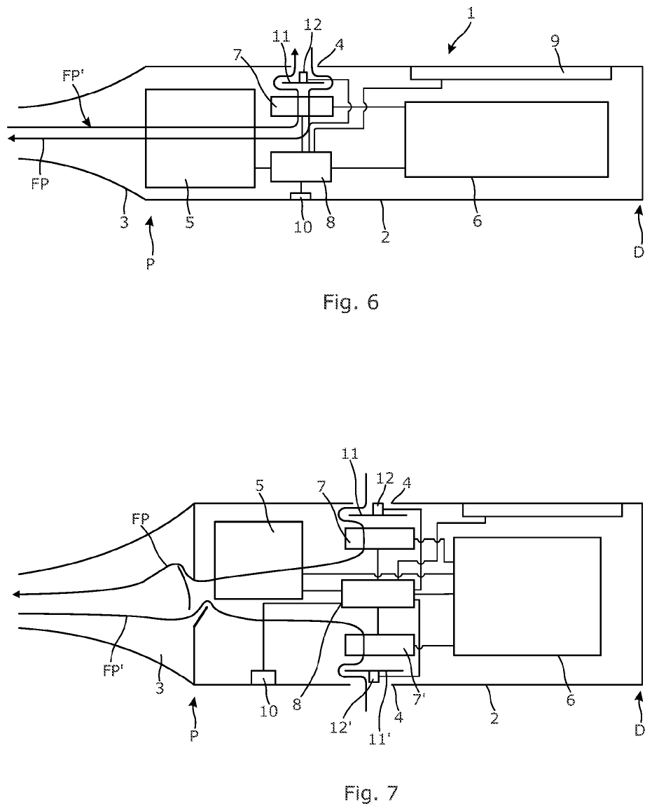

In some embodiments, and as illustrated schematically by FIG. 6, the device 1 further comprises an adjustable restrictor 11 operable to vary the resistance to air expelled out through, or drawn in through, the opening 4. The adjustable restrictor 11 comprises an occlusion mechanism 11 to vary the occlusion of the opening 4. For example, the adjustable restrictor 11 may comprise a plate 11 that overlaps the opening 4 to occlude the opening 4. The plate 11 is adjusted to vary the amount that the plate 11 overlaps the opening 4 to vary the occlusion of the opening 4. Alternatively, the plate 11 may comprise a number of apertures of varying size and be configured to rotate to selectively align said apertures with the opening 4. By changing the size of the aperture aligned with the opening 4, the plate 11 is rotated to vary the occlusion of the opening 4. It will be appreciated that other methods of varying the occlusion of the opening 4 may be employed.

The adjustable restrictor 11 may be operated manually by the user depending on their preferred resistance to inhalation or exhalation. Manual operation of the adjustable restrictor 11 may be provided electronically. For example, the adjustable restrictor 11 may be moved by an electronic actuator 12 that is connected to the battery 6 and to the control unit 8. In such examples, a control interface is provided to allow the user to manually adjust the restrictor. The control interface may be buttons provided in the housing of the device or may be integrated into the LCD display of FIGS. 5A, 5B, where, for example, said display is touch sensitive. The control interface receives the input from the user which is transmitted to the control unit 8. A signal is then generated by the control unit 8 to adjust the adjustable restrictor by activation of the actuator 12. It will be appreciated that, where manual operation of the adjustable restrictor 11 is provided, it need not require the actuator 12. Instead, the adjustable restrictor 11 may be directly manipulated by the user. For example, where the plate 11 is provide with apertures as described, the restrictor may further comprise a dial that extends from the plate 11 to the outside of the housing 2 to allow the user to rotate the plate 11 to adjust occlusion of the opening 4.

In embodiments where the adjustable restrictor 11 is provided with the actuator 12 as described, the adjustable restrictor 11 may be adjusted automatically. In other words the adjustable restrictor 11 is adjusted without direct input from the user, but as part of a control system to help the user comply with the predetermined breathing regimen. In such embodiments, the control unit 8 actively adjusts the adjustable restrictor as the user breathes through the device 1 to adjust the flow rate through the opening to keep to the predetermined flow rate of inhalation and exhalation. In such embodiments, the adjustable restrictor 11 can be said to be part of the breathing guidance means 9.

In such embodiments, the control unit 8 continuously calculates the difference between the flow rate—as output by the flow rate sensor 7—and the predetermined flow rate. The control unit 8 then sends a signal to the actuator 12 to adjust the restrictor 11 accordingly. If the control unit 8 determines that the flow rate is below the predetermined flow rate, then the control unit 8 sends a signal to the actuator 12 to operate the adjustable restrictor 11 to decrease occlusion of the opening 4. If the control unit 8 determines that the flow rate is above the predetermined flow rate, then the control unit 8 sends a signal to the actuator 12 to operate the adjustable restrictor 11 to increase occlusion of the opening 4. This process is continuous as the user inhales or exhales through the device 1. Therefore, as the user breathes through the device 1, their breathing rate is adjusted to comply with the predetermined breathing regimen. This process may be switchable so that the user can elect whether to allow the device 1 to automatically adjust the adjustable restrictor 11, or not. For example, the user may wish to control their breathing themselves and rely upon any of the visual, audio or haptic breathing guidance means 9 discussed above.

In some embodiments, as illustrated by FIG. 7, the opening 4 comprises a separate inlet 4 and outlet 4′ that form part of the inhalation and exhalation flow paths FP, FP′, respectively. Each of the inhalation and exhalation flow paths FP, FP′ comprises a one way valve 13, 13′. The one way valve 13 of the inhalation flow path FP is configured to ensure air flows only in the direction from the inlet 4 to the mouthpiece 3. The one way valve 13′ of the exhalation flow path FP′ is configured to ensure air flows only in the direction from the mouthpiece 3 to the outlet 4′. Therefore, as the user breathes through the device 1, air is inhaled along the inhalation flow path FP and exhaled along the exhalation flow path FP′. In the illustrated embodiment, the exhalation flow path FP′ is directed around the air modifier 5. It will be appreciated that, although the one way valves 13, 13′ are only shown in FIG. 7, they may also be employed in any other embodiment described herein. Specifically, where it is beneficial to cause the inhalation and exhalation flow paths FP, FP′ to diverge, a respective one way valve 13, 13′ may be used at one or other end of where the flow paths FP, FP′ diverge. This may be useful, for example, where it is necessary to divert the exhalation flow path around the air modifier 5.

Referring again to FIG. 7, the inlet and the outlet comprise separate adjustable restrictors 11, 11′. Each adjustable restrictor 11, 11′ is substantially as described above with reference to FIG. 6, comprising an occlusion mechanism to vary the occlusion of the respective inlet or outlet 4, 4′.

As described above with reference to adjustable restrictor 11 of FIG. 6, each adjustable restrictor 11, 11′ of FIG. 7 may be operated manually by the user depending on their preferred resistance to inhalation or exhalation. Again, manual operation of the adjustable restrictors can be achieved electronically, with movement effected by an electronic actuator, or by direct manipulation of a dial of the adjustable restrictors 11, 11′. Again, a control interface may be provided to allow the user to manually adjust the restrictors 11, 11′. The control interface may allow for independent adjustment of each adjustable restrictor 11, 11′ so that the user can set a resistance to inhalation separate to their selected resistance for exhalation.

Each adjustable restrictor 11, 11′ may alternatively be adjusted automatically. In other words each adjustable restrictor 11, 11′ may be adjusted without direct input from the user, but as part of a control system to help the user comply with the predetermined breathing regimen. In such embodiments, the control unit 8 actively adjusts each adjustable restrictor 11, 11′ as the user breathes through the device 1 to adjust the flow rate through the inlet and outlet 4, 4′ to keep to the predetermined flow rate of inhalation and exhalation. Each of the inlet and the outlet 4, 4′ are provided with their own flow rate sensor 7, 7′. As the user breathes through the device, the flow rate sensors 7, 7′ output the respective flow rate through the inlet and the outlet 4, 4′ to the control unit 8. The control unit 8 continuously calculates the difference between the flow rates for the inlet and the outlet 4, 4′ and respective predetermined flow rates for the inlet and outlet 4, 4′. The control unit 8 sends a signal to each actuator 12, 12′ to adjust each restrictor accordingly. If the control unit 8 determines that the flow rate for the inlet 4 is below the predetermined flow rate, then the control unit 8 sends a signal to the actuator 12 at the inlet 4 to operate the inlet adjustable restrictor 11 and decrease occlusion of the inlet 4. If the control unit 8 determines that the flow rate for the inlet 4 is above the predetermined flow rate, then the control unit 8 sends a signal to the actuator 12 at the inlet 4 to operate the inlet adjustable restrictor 11 to increase occlusion of the inlet 4. Likewise, if the control unit determines that the flow rate for the outlet 4′ is below the predetermined flow rate, then the control unit 8 sends a signal to the actuator 12′ at the outlet 4′ to operate the outlet adjustable restrictor 11′ and decrease occlusion of the outlet 4′. If the control unit 8 determines that the flow rate for the outlet 4′ is above the predetermined flow rate, then the control unit 8 sends a signal to the actuator 12′ at the outlet 4′ to operate the outlet 4′ adjustable restrictor 11′ to increase occlusion of the outlet 4′. This process of adjustment of the inlet and outlet adjustable restrictors 11, 11′ is continuous as the user inhales or exhales through the device 1. Therefore, as the user breathes through the device 1, their breathing rate is adjusted to comply with the predetermined breathing regimen. This process may be switchable so that the user can elect whether to allow the device to automatically adjust the adjustable restrictors 11, 11′, or not. For example, the user may wish to control their breathing themselves and rely upon any of the visual, audio or haptic breathing guidance means 9 discussed above.

As in above embodiments, each flow rate sensor 7, 7′ is configured to send a signal to the control unit 8 as flow is detected. The signals comprise information about the flow rate so that the control unit 8 may determine both the duration inhalation or exhalation and the flow rate of inhalation and exhalation. This means that having separate flow rate sensors 7. 7′ for the inlet and the outlet 4, 4′ does not affect the functionality of the device of FIG. 7. Indeed, the device of FIG. 7 may offer any of the functionality described above with reference to FIGS. 1 to 6 or FIGS. 8 to 13. The only difference is that information relating to inhalation is provided to the control unit 8 by the flow sensor 7 at the inlet 4, while information relating to exhalation is provided by the flow sensor 7′ at the outlet 4′, rather than said information being provided by a single flow rate sensor 7 at single opening 4.

In embodiments in which the air modifier 5 comprises a non-combustible aerosol provision system 5, the breathing guidance means 9 may be further configured to select a predetermined breathing regimen in dependence on information about aerosol generating material installed in the non-combustible aerosol provision system 5. By ‘select a predetermined breathing regiment’, it is meant that the breathing guidance means 9 is configured to encourage compliance with a predetermined breathing regimen that corresponds with a predetermined aerosol generating material. For example, the breathing guidance means 9 may be configured to encourage compliance with a first predetermined breathing regimen when a first aerosol generating material is installed in the non-combustible aerosol provision system 5 and to encourage compliance with a second predetermined breathing regimen when a second aerosol generating material is installed in the non-combustible aerosol provision system 5. The first aerosol generating material and the first predetermine breathing regimen are chosen to promote a different mood in the user than the second aerosol generating material and the second predetermine breathing regimen.

In embodiments of the device 2 comprising the non-combustible aerosol generating system 5 of FIG. 8, the breathing guidance means 9 may be further configured to select a predetermined breathing regimen in dependence on information about the aerosol former material received in the tank 81. In such embodiments, a number of different types of aerosol former material can be provided, each type of aerosol former material being configured to promote a particular mood. The breathing guidance means 9 is therefore configured to select a predetermined breathing regimen that enhances the mood promoted by the chosen aerosol former material. For example, a user can select a predetermined breathing regimen via any suitable user interface, such as via a touch sensitive screen on the device (where provided) or using an application on a mobile electronic device 201 in communication with the device 1, such as the mobile electronic device 201 shown in the system of FIG. 13. In some embodiments, the tank 81 is replaceable and provided with electronic identification means that allows the control unit 8 to determine the type of aerosol former material in the tank 81. Therefore, when such a tank 81 is installed in the device, the control unit 8 can identify the aerosol former material and select a predetermined breathing regimen automatically, i.e. in dependence on information about the aerosol former material, without any further input from the user.

In embodiments of the device 2 comprising the aerosol generating system of FIGS. 10-12, the breathing guidance means 9 may be further configured to select a predetermined breathing regimen in dependence on information about the article 40 received in the device 1. In such embodiments, a number of different types of article 40 are provided, each type of article 40 being configured to promote a particular mood. The breathing guidance means 9 is therefore configured to select a predetermined breathing regimen that enhances the mood promoted by the article 40. As above, a user may select a predetermined breathing regimen via any suitable user interface, such as via a touch sensitive screen on the device (where provided) or using an application on a mobile electronic device 201 in communication with the device. The user's selection is processed by the control unit 8 which selects a predetermined breathing regimen accordingly. In some embodiments, the article 40 is provided with electronic identification means that allows the control unit 8 to determine the type of aerosol generating material on the article 40. Therefore, when the article 40 is installed in the device, the control unit 8 can identify and select a predetermined breathing regimen automatically, i.e. in dependence on information about the aerosol generating material.

In embodiments in which the plurality of portions of aerosol generating material 44a-44f comprise at least two portions of different aerosol generating material, the device 1 may be configured to adapt the predetermined breathing regimen according to the type of aerosol generating material 44 being heated by the device 1 at any given time. The article 40 may be provided with electronic identification means that allows the control unit 8 to determine the type of aerosol generating material on the article 40. Therefore, when the article 40 is installed in the device, the control unit 8 can identify and select a predetermined breathing regimen automatically, i.e. in dependence on information about the aerosol generating material. In particular the control unit can coordinate the selection of the predetermined breathing regimen in dependence on the portion of aerosol generating material 44a-44f that is being heated at any given time.

As used herein ‘aerosol-generating material’ is a material that is capable of generating aerosol, for example when heated, irradiated or energized in any other way. Aerosol-generating material may, for example, be in the form of a solid, liquid or gel which may or may not contain an active substance and/or flavorants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavors, one or more aerosol-former materials, and optionally one or more other functional material.

The aerosol-former material may comprise one or more constituents capable of forming an aerosol. In some embodiments, the aerosol-former material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

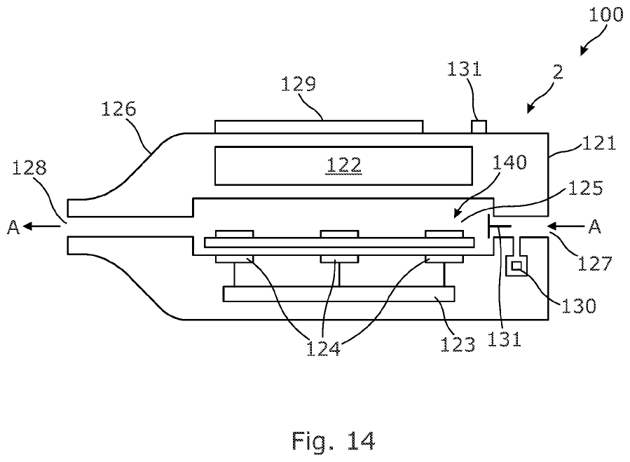

In another aspect of the invention, an aerosol provision device is provided as shown in FIGS. 14 to 16. FIG. 14 is a cross-sectional view through a schematic representation of the aerosol provision device 100. The aerosol provision device 100 comprises an outer housing 121, a power source 122, control circuitry 123, a plurality of aerosol generating components 124, a receptacle or aerosol forming chamber 125, a mouthpiece end 126, an air inlet 127, an air outlet 128, an activation button 129 and an inhalation sensor 130. The outer housing 121 is arranged such that the power source 122, control circuitry 123, aerosol generating components 124, receptacle 125 and inhalation sensor 130 are located within the outer housing 121. The outer housing 121 also defines the air inlet 127 and air outlet 128, described in more detail below. The activation button 129 is located on the exterior of the outer housing 121.

The power source 122 is configured to provide operating power to the aerosol provision device 100. The power source 122 may be any suitable power source, such as a battery. For example, the power source 122 may comprise a rechargeable battery, such as a lithium ion battery. The control circuitry 123 is suitably configured or programmed to control the operation of the aerosol provision device 100 and distribute power from the power source 122 to other components of the device 100.

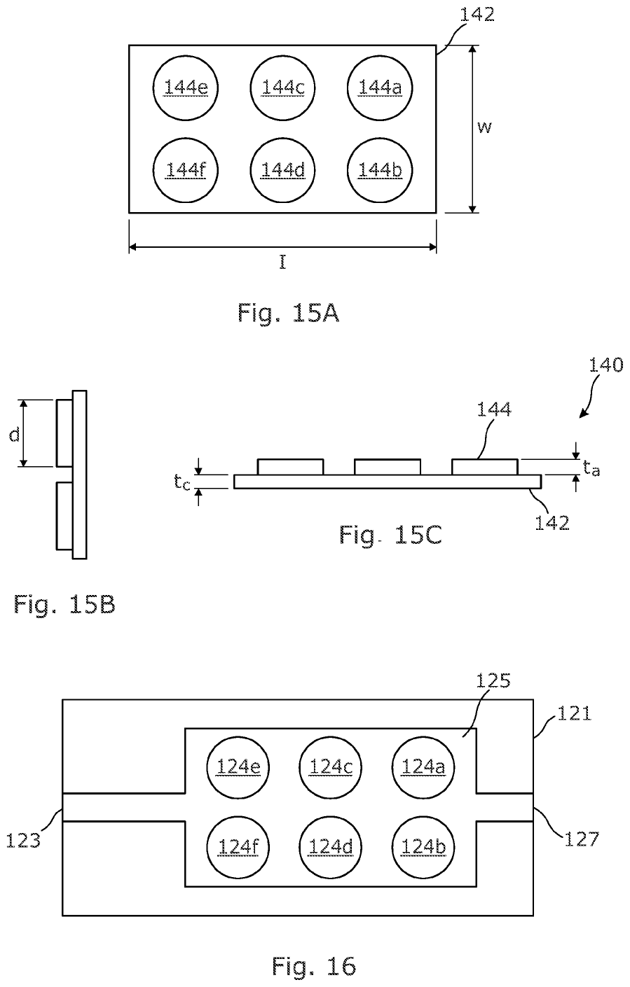

The receptacle 125 is arranged to receive an aerosol generating article 140, an example of which is shown in detail in FIGS. 15A to 15C. The aerosol generating article 140 comprises a carrier component 142 and aerosol generating material 144. FIG. 15A is a top-down view of the article 140, FIG. 15B is an end-on view along the longitudinal (length) axis of the article 140, and FIG. 15C is a side-on view along the width axis of the article 140.

In this example, the carrier component 142 is formed of card and is substantially planar. The carrier component 142 forms the majority of the article 140, and acts as a base for the aerosol generating material 144 to be deposited on. The carrier component 142 is broadly cuboidal in shape has a length l, a width w and a thickness tc as shown in FIGS. 15A to 15C. By way of example, the length of the carrier component 142 may be 30 to 80 mm, the width may be 7 to 25 mm, and the thickness may be between 0.2 to 1 mm. However, it should be appreciated that the above are exemplary dimensions of the carrier component 142, and in other implementations the carrier component 142 may have different dimensions as appropriate. In the illustrated example, the article 140 comprises a plurality of discrete portions of aerosol generating material 144 disposed on a surface of the carrier component 142. More specifically, the article 140 comprises six discrete portions of aerosol generating material 144, labelled 144a to 144f, disposed in a two by three array. However, it should be appreciated that in other implementations a greater or lesser number of discrete portions may be provided, and/or the portions may be disposed in a different array (e.g., a one by six array). In the example shown, the aerosol generating material 144 is disposed at discrete, separate locations on a single surface of the component carrier 142. The discrete portions of aerosol generating material 144 are shown as having a circular footprint, although it should be appreciated that the discrete portions of aerosol generating material 144 may take any other footprint, such as square or rectangular, as appropriate. The discrete portions of aerosol generating material 144 have a diameter d and a thickness ta as shown in FIGS. 15A to 15C. The thickness ta may take any suitable value, for example the thickness ta may be in the range of 50 μm to 1.5 mm. In some embodiments, the thickness ta is from about 50 μm to about 200 μm, or about 50 μm to about 100 μm, or about 60 μm to about 90 μm, suitably about 77 μm. In other embodiments, the thickness ta may be greater than 200 μm, e.g., from about 50 μm to about 400 μm, or to about 1 mm, or to about 1.5 mm.

The discrete portions of aerosol generating material 144 are separate from one another such that each of the discrete portions may be energised (e.g., heated) individually or selectively to produce an aerosol. In some implementations, the portions of aerosol generating material 144 may have a mass no greater than 20 mg, such that the amount of material to be aerosolized by a given aerosol generating component 124 at any one time is relatively low. For example, the mass per portion may be equal to or lower than 20 mg, or equal to or lower than 10 mg, or equal to or lower than 5 mg. Of course, it should be appreciated that the total mass of the article 140 may be greater than 20 mg.

In the described implementation, the aerosol generating material 144 is an amorphous solid. Generally, the amorphous solid may comprise a gelling agent (sometimes referred to as a binder) and an aerosol generating agent (which might comprise glycerol, for example). Optionally, the aerosol generating material 144 may comprise one or more of the following: an active substance (which may include a tobacco extract), a flavorant, an acid, and a filler. Other components may also be present as desired.

An amorphous solid aerosol generating material offers some advantages over other types of aerosolizable materials commonly found in some electronic aerosol provision devices. For example, compared to electronic aerosol provision devices which aerosolize a liquid aerosolizable material, the potential for the amorphous solid to leak or otherwise flow from a location at which the amorphous solid is stored is greatly reduced. This means aerosol provision devices or articles may be more cheaply manufactured as the components do not necessarily require the same liquid-tight seals or the like to be used.

The article 140 may comprise a plurality of portions of aerosol generating material 144 all formed form the same aerosol generating material 144 (e.g., one of the amorphous solids described above). Alternatively, the article 140 may comprise a plurality of portions of aerosol generating material 144 where at least two portions are formed from different aerosol generating material (e.g., one of the amorphous solids described above).

The receptacle 125 is suitably sized to removably receive the article 140 therein. Although not shown in FIG. 14, the device 100 may comprise a hinged door or removable part of the housing 121 to permit access to the receptacle 125 such that a user may insert and/or remove the article 140 from the receptacle 125. The hinged door or removable part of the housing 121 may also act to retain the article 140 within the receptacle 125 when closed. When the aerosol generating article 140 is exhausted or the user simply wishes to switch to a different aerosol generating article 140, the aerosol generating article 140 may be removed from the device 100 and a replacement aerosol generating article 140 positioned in the receptacle 125 in its place.

As seen in FIG. 14, the aerosol provision device 100 comprises a number of aerosol generating components 124. In this example, the aerosol generating components 124 are heating elements 124, and more specifically resistive heating elements 124. Resistive heating elements 124 receive an electrical current and convert the electrical energy into heat. The resistive heating elements 124 may be formed from, or comprise, any suitable resistive heating material, such as NiChrome (Ni20Cr80), which generates heat upon receiving an electrical current.

FIG. 16 is a cross-sectional, top-down view of the aerosol provision device 100 of FIG. 14, showing the arrangement of the heating elements 124 in more detail. In FIGS. 14 and 16, the heating elements 124 are positioned such that a surface of the heating element 124 forms a part of the surface of the receptacle 125. That is, an outer surface of the heating elements 124 is flush with the inner surface of the receptacle 125. More specifically, the outer surface of the heating element 124 that is flush with the inner surface of the receptacle 125 is a surface of the heating element 124 that is heated (i.e., its temperature increases) when an electrical current is passed through the heating element 124.

The heating elements 124 are arranged such that, when the article 140 is received in the receptacle 125, each heating element 124 aligns with a corresponding discrete portion of aerosol generating material 144. Hence, in this example, six heating elements 124 are arranged in a two by three array broadly corresponding to the arrangement of the two by three array of the six discrete portions of aerosol generating material 144 shown in FIGS. 15A to 15C. However, as discussed above, the number of heating elements 124 may be different in different implementations, for example there may be 8, 10, 12, 14, etc. heating elements 124. In some implementations, the number of heating elements 124 is greater than or equal to six but no greater than 20. More specifically, the heating elements 124 are labelled 124a to 124f in FIG. 16, and it should be appreciated that each heating element 124 is arranged to align with a corresponding portion of aerosol generating material 144 as denoted by the corresponding letter following the references 124/144. Accordingly, each of the heating elements 124 can be individually activated to heat a corresponding portion of aerosol generating material 144. While the heating elements 124 are shown flush with the inner surface of the receptacle 125, in other implementations the heating elements 124 may protrude into the receptacle 125. In either case, the article 140 contacts the surfaces of the heating elements 124 when present in the receptacle 125 such that heat generated by the heating elements 124 is conducted to the aerosol generating material 144 through the carrier component 142.

In use, the device 100 (and more specifically the control circuitry 123) is configured to deliver power to the heating elements 124 in response to a user drawing on the mouth end of the device 100.

In use, the device 100 (and more specifically the control circuitry 123) is configured to deliver power to the heating elements 124 in response to a user input. Broadly speaking, the control circuitry 123 is configured to selectively apply power to the heating elements 124 to subsequently heat the corresponding portions of aerosol generating material 144 to generate aerosol. When a user inhales on the device 100 (i.e., inhales at mouthpiece end 126), air is drawn into the device 100 through air inlet 127, into the receptacle 125 where it mixes with the aerosol generated by heating the aerosol generating material 144, and then to the user's mouth via air outlet 128. That is, the aerosol is delivered to the user through mouthpiece end 126 and air outlet 128.

The device 100 of FIG. 14 includes an activation button 129 and an inhalation sensor 130. Collectively, the activation button 129 and inhalation sensor 130 act as mechanisms for a receiving a user input to cause the generation of aerosol, and thus may more broadly be referred to as user input mechanisms. The received user input may be said to be indicative of a user's desire to generate aerosol.

The activation button 129 can be pressed by a user to start aerosol generation. The control circuitry 123 may be configured to receive signalling from the button 129 to determine if a user is pressing the button 129. If the control circuitry 123 receives this signalling, then the control circuitry 123 is configured to supply power from the power source 122 to one or more of the heating elements 124. Power may be supplied for a predetermined time period (for example, three seconds) from the signalling is detected, or in response to the length of time the signalling is detected for. In other implementations, the button 129 may be replaced by a screen type user interface.

The inhalation sensor 130 may be a pressure sensor or microphone or the like configured to detect a drop in pressure or a flow of air caused by the user inhaling on the device 100. The inhalation sensor 130 is located in fluid communication with the air flow pathway (that is, in fluid communication with the air flow path between inlet 127 and outlet 128). In a similar manner as described above, the control circuitry 123 may be configured to receive signalling from the inhalation sensor and to use this signalling to determine if a user is inhaling on the aerosol provision device 100. If the control circuitry 123 receives this signalling, then the control circuitry 123 is configured to supply power from the power source 122 to one or more of the heating elements 124. Power may be supplied for a predetermined time period (for example, three seconds) from the moment inhalation is detected, or in response to the length of time the inhalation is detected for.

In the described example, both the button 129 and inhalation sensor 130 detect the user's desire to begin generating aerosol for inhalation. The control circuitry 123 may be configured to only supply power to the heating element 124 when signalling from both the button 29 and inhalation sensor 130 are detected. This may help prevent inadvertent activation of the heating elements 124 from accidental activation of one of the user input mechanisms. However, in other implementations, the aerosol provision device 100 may have only one of a button 129 and an inhalation sensor 130.

These aspects of the operation of the aerosol provision device 100 (i.e. puff detection and touch detection) may in themselves be performed in accordance with established techniques (for example using conventional inhalation sensor and inhalation sensor signal processing techniques and using conventional button sensor signal processing techniques).

In some implementations, in response to detecting the signalling from either one or both of the touch-sensitive panel 129 and inhalation sensor 130, the control circuitry 123 is configured to sequentially supply power to each of the individual heating elements 124. More specifically, the control circuitry 123 is configured to sequentially supply power to each of the individual heating elements 123 in response to a sequence of detections of the signalling received from either one or both of the touch-sensitive panel 129 and inhalation sensor 130. For example, the control circuitry 123 may be configured to supply power to a first heating element 124 of the plurality of heating elements 124 when the signalling is first detected (e.g., from when the device 100 is first switched on). When the signalling stops, or in response to the predetermined time from the signalling being detected elapsing, the control circuitry 123 registers that the first heating element 124 has been activated (and thus the corresponding discrete portion of aerosol generating material 144 has been heated). The control circuitry 123 determines that in response to receiving subsequent signalling from either one or both of the button 29 and inhalation sensor 130 that a second heating element 124 is to be activated. Accordingly, when the signalling from either one or both of the button 129 and inhalation sensor 130 is received by the control circuitry 123, the control circuitry 123 activates the second heating element 124. This process is repeated for remaining heating elements 124, such that all heating elements 124 are sequentially activated.

Effectively, this operation means that for each inhalation a different one of the discrete portions of aerosol generating material 144 is heated and an aerosol generated therefrom. In other words, a single discrete portion of aerosol generating material is heated per user inhalation.

The device of FIG. 14 further comprises an adjustable restrictor 131 at the inlet 127, the adjustable restrictor 131 is configured to vary the resistance to draw. That is to say, the adjustable restrictor 131 is configured to adjust the effort required to draw air in through the inlet 127 and along the air flow pathway. The adjustable restrictor 131 can be configured to be manually adjustable or automatically adjustable, as will be explained further below.

The adjustable restrictor may comprise any mechanism to vary the resistance to draw. For example, the adjustable restrictor 131 may comprise an occlusion mechanism 131 to vary the occlusion of the inlet 127. For example, the adjustable restrictor 131 may comprise a plate that overlaps the inlet 127 to occlude the inlet 127. The plate being adjustable to vary the amount that the plate overlaps the inlet 127 to vary the occlusion of the inlet 127. Alternatively, the plate may comprise a number of apertures of varying size and be configured to rotate to selectively align said apertures with the inlet 127. By changing the size of the aperture aligned with the inlet 127, the plate is rotated to vary the occlusion of the inlet 127. It will be appreciated that other methods of varying the occlusion of the inlet 127 may be employed.

The adjustable restrictor 131 may be operated manually by the user depending on their preferred resistance to draw. Manual operation of the adjustable restrictor 131 may be provided electronically. For example, the adjustable restrictor 131 may be moved by an electronic actuator that is connected to the battery 122 and to the control circuitry 123. In such examples, a control interface is provided to allow the user to manually adjust the restrictor 131. The control interface may be buttons provided in the housing 121 of the device 100 or may be integrated into a touch sensitive display. The control interface receives the input from the user which is transmitted to the control circuitry 123. A signal is then generated by the control circuitry 123 to adjust the adjustable restrictor 131 by activation of the actuator. It will be appreciated that, where manual operation of the adjustable restrictor 131 is provided, it need not require the actuator. Instead, the adjustable restrictor 131 may be directly manipulated by the user. For example, where the plate is provide with apertures as described, the restrictor 131 may further comprise a dial that extends from the plate to the outside of the housing 121 to allow the user to rotate the plate to adjust occlusion of the inlet 127.

In embodiments where the adjustable restrictor 131 is provided with the actuator as described, the adjustable restrictor 131 may be adjusted automatically. In other words the adjustable restrictor 131 is adjusted without direct input from the user, but as part of a control system to help maintain a predetermined draw resistance. In such embodiments, the control circuitry 123 actively adjusts the adjustable restrictor 131 as the user draws on the device 100 to adjust the pressure drop across the inlet 127 to keep to the predetermined draw resistance. For example, the inhalation sensor determines the pressure at the inlet 127 and continuously relays this to the control circuitry 123 which calculates a difference between the pressure at the inlet and a predetermined inlet pressure indicative of a predetermined draw resistance. The control circuitry 123 then sends a signal to the actuator to adjust the restrictor 131 accordingly. If the control circuitry 123 determines that the pressure at the inlet 127 is below the predetermined pressure, then the control circuitry 123 sends a signal to the actuator to operate the adjustable restrictor 131 to decrease occlusion of the inlet 127. If the control circuitry 123 determines that the pressure at the inlet 127 is above the predetermined pressure, then the control circuitry 123 sends a signal to the actuator to operate the adjustable restrictor 131 to increase occlusion of the inlet 127. This process is continuous as the user draws on the device 100.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in the future.

1. A device comprising a mouthpiece and an opening in fluid communication with the mouthpiece, wherein the device is configured to allow a user to breathe through the mouthpiece by drawing air in through the opening and expelling air out through the opening, the device further comprising:
breathing guidance means configured to encourage compliance with a predetermined breathing regimen; and
an air modifier, wherein the air modifier comprises any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

2. A device according to claim 1, wherein the breathing guidance means comprises an adjustable restrictor operable to vary the resistance to air expelled out through the opening.

3. A device according to claim 2, wherein the adjustable restrictor is operable to vary the resistance to air drawn in through the opening.

4. A device according to claim 2 or 3, wherein the device further comprises a control unit and a flow rate sensor, wherein the flow rate sensor is configured to send a signal to the control unit indicative of a flow rate of air expelled out through the opening and/or inhaled in through the opening.

5. A device according to claim 4, wherein the flow rate sensor is configured to send a signal to the control unit indicative of the duration of an inhalation or exhalation event.

6. A device according to claim 4, wherein the adjustable restrictor is operable to vary the resistance to air expelled out through the opening in response to a signal from the control unit.

7. A device according to claim 6, wherein the adjustable restrictor comprises an occlusion mechanism to occlude the opening, wherein the occlusion mechanism is adjustable to vary the occlusion of the opening.

8. A device according to claim 7, wherein the opening comprises a separate inlet and outlet, wherein the inlet and outlet each comprise a one-way valve, the valve of the inlet being configured to allow air to flow only in a direction into the device and the valve of the outlet being configured to allow air to flow only in a direction out of the device.

9. A device according to claim 8, wherein the adjustable restrictor comprises an inlet occlusion mechanism and an outlet occlusion mechanism, each of the inlet and outlet occlusion mechanisms being independently adjustable to vary the occlusion of the inlet and the outlet, respectively.

10. A device according to claim 9, wherein the flow rate sensor comprises an inlet flow rate sensor and an outlet flow rate sensor, the inlet flow rate sensor being configured to send a signal to the control unit indicative of a flow rate of air drawn in through the inlet and the outlet flow rate sensor being configured to send a signal to the control unit indicative of a flow rate of air expelled out through the outlet.

11. A device according to claim 10, wherein the inlet flow rate sensor is configured to send a signal to the control unit indicative of the duration of an inhalation event and/or wherein the outlet flow rate sensor is configured to send a signal to the control unit indicative of the duration of an exhalation event.

12. A device according to claim 11, wherein the inlet occlusion mechanism is configured to adjust the occlusion of the inlet in response to a signal from the control unit and/or wherein the outlet occlusion mechanism is configured to adjust the occlusion of the outlet in response to a signal from the control unit.

13. A device according to claim 12, wherein the inlet flow rate sensor flow rate sensor selected from a list of sensors comprising a microphone, a volumetric flow meter or a mass flow meter; and/or wherein the outlet flow rate sensor flow rate sensor selected from a list of sensors comprising a microphone, a volumetric flow meter or a mass flow meter.

14. A device according to claim 4, wherein the breathing guidance means comprises haptic feedback means and/or wherein the breathing guidance means comprises visual display means communicable with the control unit.

15. A device according to claim 14, wherein the haptic feedback means or the visual display means are configured to alert a user if the flow rate of air drawn in through the inlet and/or expelled out through the outlet falls outside a predetermined range.

16. A device according to claim 14, wherein the haptic feedback means or the visual display means are configured to alert a user if the duration of an inhalation and/or exhalation event falls outside a predetermined range.

17. A device according to any of claims 1 to 3, wherein the aerosol generator is configured to vaporize an aerosol former material.

18. A device according to claim 17, wherein the aerosol former material comprises nicotine.

19. A device according to any of claims 1 to 3, wherein the aerosol generator is configured to generate an aerosol from an article comprising a plurality of aerosol generating regions, the device comprising one or more heating elements.

20. A device according to claim 19, wherein, in use, an aerosol generating article comprising a plurality of aerosol generating regions is located so that one or more aerosol generating regions are located adjacent a heating element of the one or more heating elements.

21. A device according to any of claims 1 to 3, wherein the aerosol generator comprises a supply of aerosol.

22. A device according to claim 21, wherein the supply of aerosol comprises purified air.

23. A device according to claim 5, wherein the control unit is configured to transmit the signal indicative of flow rate and/or the signal indicative of the duration of inhalation or exhalation events to a mobile electronic device.

24. A system comprising a handheld device and a mobile electronic device;
wherein the handheld device comprises a mouthpiece and an opening in fluid communication with the mouthpiece, the device being configured to allow a user to breathe through the mouthpiece by drawing air in through the opening and expelling air out through the opening, and wherein the device further comprises a flow rate sensor configured to generate data representing a flow rate of air expelled out through the opening, and breathing guidance means configured to encourage compliance with a predetermined breathing regimen;
wherein the mobile device is communicable with the handheld device and comprises computer code, the mobile device being configured to execute the computer code to receive data from the handheld device representing a flow rate of air expelled out through the opening; and
wherein the handheld device further comprises an air modifier, the air modifier comprising any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

25. A system according to claim 24, wherein the mobile device is further configured to execute the computer code to remotely operate the breathing guidance means.

26. A system according to claim 25, wherein the breathing guidance means comprises an adjustable restrictor to restrict flow through the opening and wherein the mobile device is further configured to execute the computer code to remotely operate the adjustable restrictor.

27. A system according to claim 25 or claim 26, wherein the mobile device is configured to execute the computer code to display information to the user to prompt the user to control their breathing in accordance with a predetermined breathing regimen.

28. A system according to claim 25, wherein the mobile device is configured to execute the computer code to remotely operate the breathing guidance means in response to data received from the flow rate sensor.

29. A method of operating a system comprising a handheld device and a mobile device;
wherein the handheld device comprises a mouthpiece and an opening in fluid communication with the mouthpiece, the device being configured to allow a user to breathe through the mouthpiece by drawing air in through the opening and expelling air out through the opening, and wherein the device further comprises a flow rate sensor configured to generate data representing a flow rate of air expelled out through the opening, and breathing guidance means configured to encourage compliance with a predetermined breathing regimen, the method comprising,
at the handheld device:
utilizing the flow rate sensor to generate data representing a flow rate of air expelled out through the opening,
transmitting the data representing the flow rate to the mobile device; and
at the mobile device:
receiving the data representing the flow rate of air expelled out through the opening;
wherein the handheld device further comprises an air modifier, the air modifier comprising any of:
an air ionizer configured to ionize air drawn in through the opening;
an aerosol generator for generating an aerosol for entrainment in air drawn in through the opening;
a heater to heat air drawn in through the opening; or
means to cool air drawn in through the opening.

30. A method according to claim 29, further comprising, at the mobile device, remotely operating the breathing guidance means of the handheld device.

31. A method according to claim 30, wherein the breathing guidance means comprises an adjustable restrictor to restrict flow through the opening and wherein the method further comprises, at the mobile device, remotely operating the adjustable restrictor.

32. A method according to claim 30 or claim 31, further comprising, at the mobile device, displaying information to the user to prompt the user to control their breathing in accordance with a predetermined breathing regimen.

33. A non-combustible aerosol provision device comprising an inlet and a mouthpiece in fluid communication with the inlet, wherein the device further comprises an adjustable restrictor configured to vary a draw resistance across the inlet.

34. A device according to claim 33 further comprising a power source and an aerosol generating component configured to draw power from the power source to generate an aerosol by heating an article comprising aerosol generating material.

35. A device according to claim 34 further comprising an aerosol forming chamber configured to receive an article comprising an aerosol generating material.

36. A device according to claim 35 wherein the aerosol forming chamber is configured to receive an article comprising a substantially planar carrier component and a plurality of discrete portions aerosol generating material provided on the carrier component.

37. A device according to claim 36, wherein the aerosol generating component comprises a plurality of aerosol generating components and each aerosol generating component of the plurality of aerosol generating components is configured to heat one of the discrete portions of aerosol generating material of an article received in the device.
